(19) United States
(12) Patent Application Publication
(10) Pub. No.: US 2015/0174185 A1
(43) Pub. Date: Jun. 25, 2015

(54) VIRAL TRAPPERS

(71) Applicant: BHASKARA RAO KATRAGADDA, (US)

(72) Inventors: Bhaskara Rao Katragadda, Tenali (IN); P. Babu Vadlamudi, Chelsea, MI (US)

(73) Assignee: KATRADDA BHASKARA RAO

(21) Appl. No.: 14/392,009

(22) PCT Filed: Mar. 18, 2013
(86) PCT No.: PCT/IN2013/000165

§ 371 Date: Jan. 15, 2015

(30) Foreign Application Priority Data
Jul. 16, 2012 (IN) 2877/CHE/2012

(51) Int. Cl. A61K 36/48

(52) U.S. Cl. CPC A61K 36/48

(57) ABSTRACT

Viral trappers is our invention consisting of an extract of tephrosia Purpurea axial parts supplemented with immuno boosters such as chromium and zinc. Viral trappers is a very effective tool to fight intractable HIV/AIDS. Viral trappers is also an effective curative for several other viral diseases such as common cold, cold sores, hepatitis B and other opportunistic infections.

The mechanism of action of viral trappers is not known but may involve compounds like pseudo semi glabrin-semi (-) glabrinal, and semiglabrin. Viral trappers from Tephrosia Purpurea extracts therefore, contain powerful antiviral compounds or morphological structures which are also very effective against several other ailments.

Patients with HIV/AIDS recovered completely to normal, good health and productive lives even after 10 years. Therefore we are seeking an international patent for Viral trappers.

The axial parts of Tephrosia Purpurea were dried and extracted with water and filtered (coarse). This was then supplemented with other ingredients, preparing pharmaceutical dosage forms.

### FIELD OF INVENTION

Pharmaceuticals HIV/AIDS and related infections.

### BACKGROUND

We have a medical shop (Pharmacy) several People come to discuss their health problems with us. Recently it has come to our attention that in a nearby village doctors are treating some of their patients with oral cavity infections by asking them to chew on the arial parts of the plant “Vempali”. This seems to cure infections of the mouth including viral infections.

By referring to an ayurvedic compendium we discovered that the vempalli plant (Tephrosia Purpurea) has been in use for centuries treating several diseases. Therefore, we reasoned that vempalli (Tephrosia Purpurea) may contain powerful antiviral compound (s) against HIV/AIDS Virus.

A friend who is an HIV/AIDS Patient, receiving ART therapy was not responding to Govt issued anti retroviral drugs, asked us for help because, one of us is a pharmacist with a B. Pharma Degree. Our friend was in a DIRE situation and having information about Tephrosia purpurea, we recommended that he use, T. Purpurea arial parts in his food.

He actually collected aerial parts of the T. Purpurea, dried them in the sun, powdered it in a blender, mixed it with milk and drank it for 2 to 3 months. After this his health is completely back to normal and he is now a productive member of our community.

Our friend's wife, also HIV/AIDS Patient, on ART therapy used T. Purpurea arial parts powder mixed with milk. She also improved dramatically and recovered completely (CD4 count improved drastically with reduced Viral load). She suffered severe diarrhea going to bathroom about 10 times an hour recovered from this ailment also.

Similar results were observed with two other people who had been diagnosed with HIV/AIDS. In all these cases it was observed that the HIV related infections such as colds, influenza, TB, Herpes etc were also eliminated.

Therefore, it tells us that Tephrosia Pupurea leaves contain powerful anti viral compound(s) which can be harvested to treat several viral infections. Hence, we developed a formula from the T. Purpurea arial parts extract along with known immunoboosters containing chromium and Zinc.

Therefore, we are interested in pursing this T. Purpurea as a viral trapper for intractable HIV/AIDS and other HIV related illnesses.

### OBJECT OF INVENTION

Anti retro viral drugs effective in some cases of HIV/AIDS, they are expensive and not so effective in all cases. So the formulation we have developed an effective preparation against HIV/AIDS and other viral diseases. Tephrosia purpurea and other Tephrosia species of plants have a long history. in Ayurvedic medicine against several diseases. Even though several therapeutic activities were observed and recorded against various ailments, no specific information is available with regards to modern viral diseases such as HIV/AIDS. This particular patent application containing the very specific formulation addresses this problem.

### STATEMENT OF INVENTION

Also to earn money so that we can pursue further research in this and related health fields. This particular discovery/invention is a welcome addition to the range of anti retroviral drugs to fight HIV/AIDS, and other viral diseases such as common cold, influenza, diarrhea etc. This particular invention can be tried on other less common viral diseases such as meningitis, herpes, hepatitis A, B & C, warts. Therefore we are anxious to obtain a patent for this discovery of viral trappers, and the formulation we developed.

### DETAILED DESCRIPTION

Composition of the virus trapper(s)
1. Tephrosia Purpurea leaves and other arial parts when applicable.
2. Chromium as chromium picolinate.
3. Zinc sulphate monohydrate
4. Vits B6, B12, and Folic acid
5. Pseudo Semiglabrin, Semiglabrin, semiglabrinal may be present.
6. Preservatives, approved colours and natural flavors.

When leaves were used they were dried in the sun and powdered in a blender (mixer) and the resulting powder, one tea spoonful, stirred in a glass of milk. When extract was made in a blender with water the extract was then filtered through a coarse filter. When Tephrosia purpurea plant parts other than leaves were used, the green portions or the flowers were blended with water and filtered through a coarse filter. The aqueous extract is then drunk as is or mixed with milk and used.

Depending upon the availability of raw material (T. Purpurea Leaves), cost and other difficulties we will alter the formula without sacrificing the quality of the final product. This will be done at the manufacturing level as per the market demand and prepare pharmaceutical dosage forms of all types.

### SUMMARY

1. We have identified and developed a plant product specifically for viral load trapping.

2. Even though we are not sure of its mechanism of viral trapping ability. It does appear to involve 1) Pseudo Semiglabrin, 2) Semiglabrin and 3) semiglabrinal and not similar to that of the anti retroviral drugs.

3. Perhaps. T. Purpurea extract seems to destroy the virus in some unknown fashion and thus acting as a curative for several viral diseases.

4. No adverse side effects were noticed in people who tried this-extract for treating HIV/AIDS or other viral infections.

Therefore, the formula we described here appears to be a welcome addition to the arsenal of antiviral drugs in treating HIV/AIDS and related infectious diseases.

1. Tephrosia Purpurea leaves contain one or more chemicals or morphological structures which are very active in trapping viruses.

2. These chemicals are very effective against retroviral diseases such as HIV/AIDS and restore the sufferers to normal, good health.

3. Tephrosia Purpurea leaf extract is a possible remedy for hepatitis B and perhaps, other hepatitis viruses A&C.

4. T. Purpurea extract is also a curative for common cold and influenza viruses.

5. Tephrosia purpurea leaf extracts arc able to cure people who suffer from chronic diarrhea.

6. These extracts also are effective in treating (curing) viral infections of the mouth.

7. T. Purpurea leaf extracts are also very effective against opportunistic infectious diseases associated with primary viral infections such as HIV/AIDS.

8. Finally we would like to claim that the formula we described is effective as a cure for a) HIV/AIDS, b) Common cold/influenza/cold sores, c) viral hepatitis and, d) chronic diarrhea.
